(19) United States
(12) Patent Application Publication
(10) Pub. No.: US 2026/0159548 A1
(43) Pub. Date: Jun. 11, 2026

(54) HIGH-PURITY ECHINOCANDIN DRUG IMPURITY AS WELL AS PREPARATION METHOD AND APPLICATION THEREOF

(71) Applicant: SHANGHAI TECHWELL BIOPHARMACEUTICAL CO., LTD, Shanghai (CN)

(72) Inventors: Rongguo SHI, Shanghai (CN); Min CHEN, Shanghai (CN); Yan LI, Shanghai (CN); Xiaoming JI, Shanghai (CN)

(73) Assignee: SHANGHAI TECHWELL BIOPHARMACEUTICAL CO., LTD, Shanghai (CN)

(21) Appl. No.: 18/723,724

(22) PCT Filed: Sep. 28, 2023
(86) PCT No.: PCT/CN2023/122704

§ 371 Date: Jun. 24, 2024

(30) Foreign Application Priority Data
Jul. 31, 2023 (CN) 202310956462.X

(51) Int. Cl. C07K 7/56; C07K 1/18; C07K 1/22

(52) U.S. Cl. CPC C07K 7/56; C07K 1/18; C07K 1/22

(57) ABSTRACT

The present invention provides a high-purity echinocandin drug impurity and a preparation method thereof, the structure of the echinocandin drug impurity is as shown in Formula I, and the purity of the echinocandin drug impurity is greater than 90%. The high-purity impurity can be used for qualitative and quantitative analysis of impurities in the production of micafungin sodium, thereby providing technical support for subsequent quality research on micafungin sodium and improving the quality standard of micafungin.

### CROSS REFERENCE TO RELATED APPLICATIONS

The present application claims priority to Chinese Patent Application No. 202310956462.X, entitled “HIGH-PURITY ECHINOCANDIN DRUG IMPURITY AS WELL AS PREPARATION METHOD AND APPLICATION THEREOF”, and filed to the China National Intellectual Property Administration on Jul. 31, 2023, the entire contents of which are incorporated herein by reference.

### TECHNICAL FIELD

The present application relates to the technical field of medicine, in particular to a high-purity echinocandin drug impurity as well as a preparation method and an application thereof.

### BACKGROUND

Micafungin sodium is a new generation of echinocandin antifungal drugs, mainly used for the treatment of fungemia, respiratory mycosis, gastrointestinal mycosis caused by Aspergillus and Candida, and also for the prevention of Aspergillus and Candida infections in hematopoietic stem cell patients. It is a type of echinococcin antifungal agent that can inhibit the synthesis of beta (1,3)-glucan in filamentous fungi and yeasts. In recent years, the morbidity and mortality of fungal infections have also increased, especially for critically ill patients. Micafungin sodium, as a type of echinococcin drug with good antifungal activity, is the preferred drug for treating infections caused by Candida or Aspergillus. It has good therapeutic effects and little impact on human cells, with low toxicity and high efficiency in clinical practice.

In the drug development process, the analysis of impurities is crucial. Impurities are closely related to the quality, safety and stability of drugs. Preparation and structural confirmation of impurities can help us understand the pathways by which impurities are produced, and provide a basis for the improvement of drug synthesis routes and production processes. Impurities can be used as reference substance for establishing analytical methods in pharmaceutical quality control. For pharmaceutical manufacturers, strict control of impurities is required.

Micafungin sodium is a semi-synthetic antibacterial drug. It can be obtained from the fermentation intermediate FR901379 by enzymatically removing the fatty acid side chain to obtain FR179642, and then chemical synthesized by condensation reaction with the active side chain. During the fermentation process of FR901379, impurities with a similar structure to the mother core of FR901379 will be produced. The structure of this type of impurity is highly similar to that of micafungin sodium, and the removal effect is poor in the subsequent purification process. Therefore, the impurity content needs to be controlled during the fermentation and acyltransferase removal of fatty acid process stages. After studying the commercially available pharmaceutical preparations, it was found that there are multiple impurities in the HPLC spectra of the preparation products. The content of the impurities in the raw materials is relatively low, and the enrichment and purification is very difficult. In addition, echinocandins have multiple chiral centers and multiple peptide bonds and other functional groups that are unstable to acid, alkali, heat, and light. Therefore, it is difficult to obtain such impurity products with high-purity.

Relevant research on impurities in echinocandins can be used for qualitative and quantitative analysis of impurities in the production of micafungin sodium, thereby providing technical support for subsequent quality research on micafungin sodium and improving the quality standard of micafungin sodium. Therefore, there is currently a need to provide an efficient and convenient method for the separation and preparation of high-purity echinocandin drug impurity.

### SUMMARY OF THE INVENTION

In the present application, an echinocandin drug impurity represented by Formula II with a highly similar structure to that of micafungin sodium was separated and prepared from the fermentation broth of FR901379, which was removed fatty acids under the action of acyltransferase to form a compound of Formula III, a pure product of compound of Formula III was obtained by efficiently purification and separation under suitable conditions. High-purity drug impurities of echinococcin as shown in Formula I were prepared and purified. The present application adopts the following technical solution:

An echinocandin drug impurity, wherein a structure of the echinocandin drug impurity is shown in Formula I, and the echinocandin drug impurity has a purity of greater than 90%;

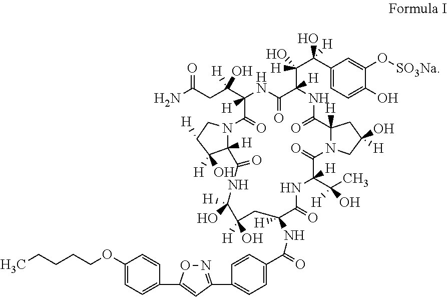

Optionally, the purity refers to HPLC purity.

A method for preparing the echinocandin drug impurity described above, wherein the method comprises the following steps:
step 1) adding an organic solvent to a microbial fermentation broth containing a compound of Formula II, mixing well and then filtering to obtain a filtrate, and enriching the filtrate by using a first adsorption resin to obtain the compound of Formula II;

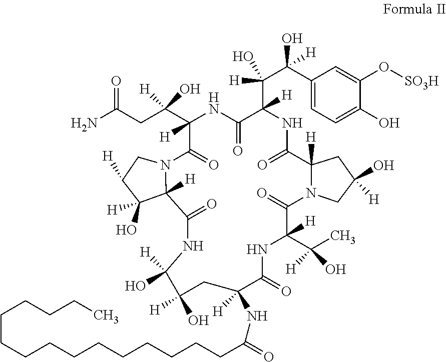

step 2) reacting the enriched compound of Formula II with an acyltransferase to obtain a compound of Formula III;

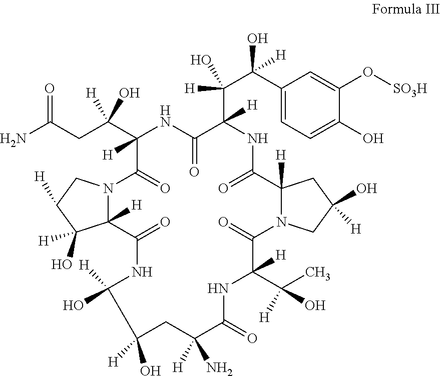

step 3) reacting the compound of Formula III obtained with the side chain of micafungin sodium to obtain the echinocandin drug impurity.

Optionally, the microbial fermentation broth in step 1) is a fermentation broth of FR901379.

Optionally, the microbial fermentation broth in step 1) is obtained from fermentation of Coleophoma sp.

Optionally, the organic solvent is selected from at least one of methanol, ethanol, propanol, acetone and butanone.

Optionally, a volume ratio of the organic solvent to the microbial fermentation broth is in a range from 1% to 40%.

Optionally, the first adsorption resin is a non-polar aromatic adsorption resin formed by polymerization of styrene and divinylbenzene.

Optionally, the first adsorption resin is at least one of AD-1, HP20, HP20SS, HP30, SP-825, SP-850, SP207 and CHP20.

Optionally, the acyltransferase is a carrier-loaded immobilized acyltransferase.

Optionally, the carrier is selected from one or a combination of silicon dioxide, aluminum trioxide, epoxy bonded with polymethyl methacrylate as the matrix, and amino functional groups.

Optionally, reacting the enriched compound of Formula II with an acyltransferase in step 2) further comprises a purification step, wherein the purification step comprises: adsorption using a second adsorption resin, and elution using an organic solvent aqueous solution at a temperature ranging from 10° C. to 30° C.

Optionally, the second adsorption resin is a macroporous adsorption resin, which is at least one of the resins SP207, SP207SS, UniPS10, UniPS20 and UniPS50 using styrene and divinylbenzene as matrix.

Optionally, the organic solvent in the organic solvent aqueous solution is selected from at least one of methanol, ethanol, acetone and acetonitrile.

Optionally, the volume percentage of organic solvent in the organic solvent aqueous solution is in a range from 1% to 90%.

Optionally, the elution is performed at a temperature in a range from 15° C. to 25° C.

Optionally, the pH of the purified collection solution needs to be adjusted to 1.0 to 6.0.

Optionally, the optimal pH of the purified collection solution is adjusted to 3.0 to 4.0.

Optionally, the compound of Formula III is reacted with the side chain of micafungin sodium at a temperature ranging from −20° C. to 10° C.

Optionally, the compound of Formula III is reacted with the side chain of micafungin sodium in an organic solvent selected from at least one of toluene, N,N-Dimethylformamide (DMF), N,N-dimethylacetamide (DME), dimethyl sulfoxide (DMSO), tetrahydrofuran (THF) and dichloromethane.

Optionally, after the completion of reacting the compound of Formula III with the side chain of micafungin sodium, purification is performed, and the purification is performed by using ion exchange resin, macroporous adsorption resin, and silica gel in sequence.

Optionally, the ion exchange resin is a strongly acidic cation exchange resin, and the ion exchange resin is selected from at least one of UBK530, UBK550 and UBK555.

Optionally, the macroporous adsorption resin is selected from at least one of HP20, HP20SS, SP70, SP700, SP825L, SP850, CHP20 and CHP55.

Optionally, the silica gel is selected from at least one of pure silica gel, amino-bonded silica gel, diol-bonded silica gel and amide-bonded silica gel.

Optionally, the silica gel is selected from at least one of UniSil® HILIC silica gel (nano micro), Chromatorex ARG Silica Fuji, Click XIon silica gel (Huapu Xinchuang), and Inertsil HILIC silica gel (Shimadzu, Japan).

Optionally, the organic solvent in the acidic organic solvent aqueous solution is selected from one of methanol, ethanol, acetone and acetonitrile.

Optionally, the purification using silica gel comprises adsorption using a silica gel column, and elution using an acidic organic solvent aqueous solution with a pH value ranging from 2.0 to 7.0.

Optionally, during the adsorption using a silica gel column, the proportion of organic solvent in the sample solution should not exceed 5%.

Optionally, the volume percentage of organic solvent and water in the acidic organic solvent aqueous solution is in a range from 20% to 100%.

Optionally, the volume percentage of organic solvent and water in the acidic organic solvent aqueous solution is in a range from 50% to 100%.

Optionally, the acidic organic solvent aqueous solution contains at least one of formic acid, acetic acid, hydrochloric acid, phosphoric acid and trifluoroacetic acid.

Optionally, a pH value of the acidic organic solvent aqueous solution is in a range from 3.0 to 5.0.

Optionally, the purity of the prepared compound of Formula I is greater than 90%.

The present application also proposes application of the above-mentioned cyclic peptide compound in the quality control of echinocandin drugs.

As a specific embodiment, the chemical name of the echinocandin drug impurity provided by the present application is sodium 5-[(1S,2S)-2-[(3S,6S,9S,11R,15S,18S,20R,21R,24S,25S)-3-[(R)-2-carbamoyl-1-hydroxyethyl]-11,20,21,25-tetrahydroxy-15-[(R)-1-hydroxyethyl]-2,5,8,14,17,23-hexoxo-18-[4-[5-(4-pentoxyphenyl) isoxazol-3-yl]benzoylamino]-1,4,7,13,16,22-hexaazatricyclo[22.3.0.0^{9,13}]heptacos-6-yl]-1,2-dihydroxyethyl]-2-hydroxyphenyl sulfate, the structure is shown in Formula I.

A method for preparing the compound of Formula I comprises the following steps:
(a) fermentation to obtain fermentation broth containing FR901379 impurity (Formula II), and extraction and enrichment;

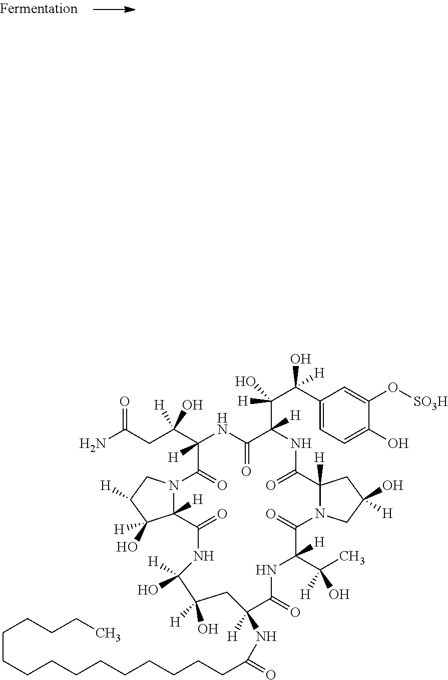

(b) conversion using acyltransferase to obtain a component containing the FR179642 impurity (Formula III), and purification and isolation to obtain the compound of Formula III; and

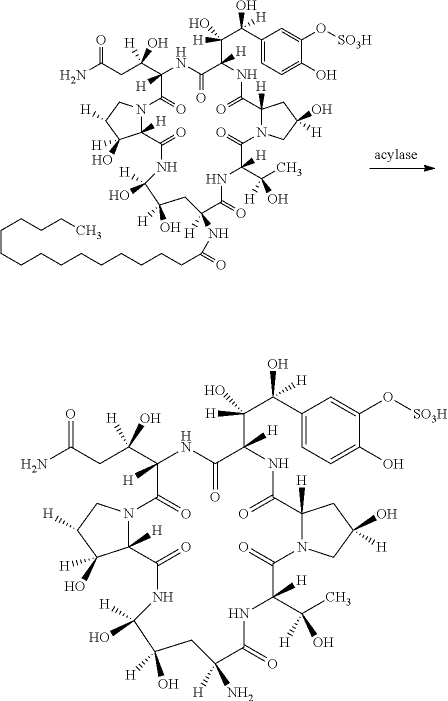

(c) preparation by condensation reaction of FR179642 impurity (Formula III) with the side chain of micafungin sodium, and purification to obtain micafungin sodium drug impurity (Formula I).

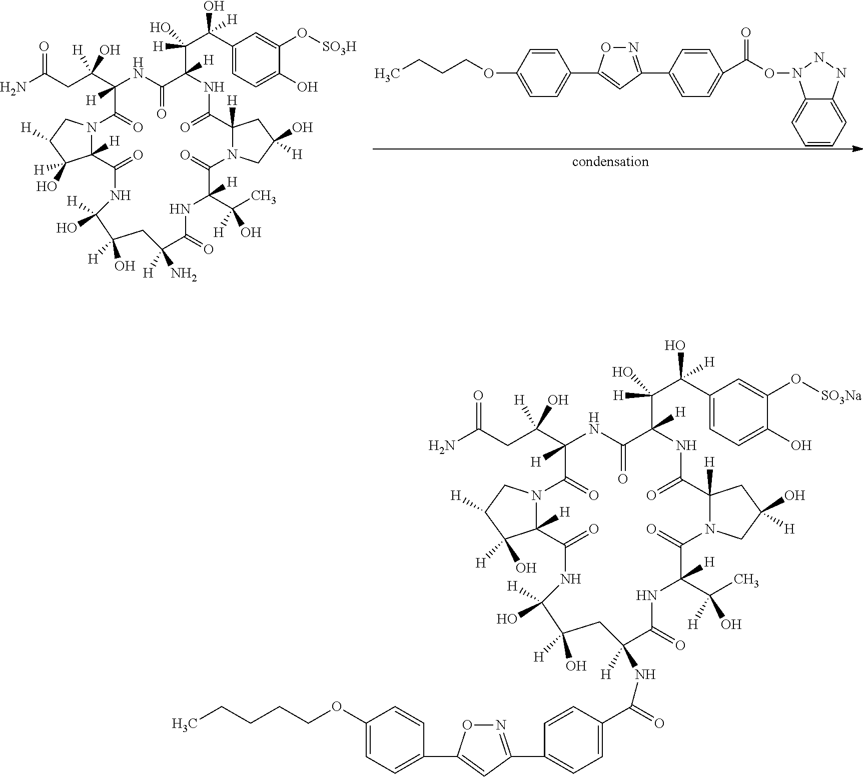

The fermentation in step (a) refers to patent CN102618604B, using Coleophoma sp as the fermentation strain, the main components of the culture medium include cottonseed powder, mannitol, sodium glutamate, and proline, and cultured at a temperature ranging from 23° C. to 28° C. for 6 days to 8 days.

As a further improvement of the above preparation method, the purification described in step (b) comprises:
1) adsorption using macroporous adsorption resin; and
2) elution using a certain proportion of organic solvent at low temperature.

As a further improvement of the above preparation method, the macroporous adsorption resin in step 1) is an aromatic adsorption resin formed by polymerization of styrene/divinylbenzene, selected from one or a combination of SP207, SP207SS, UniPS10, UniPS20 and UniPS50.

As a further improvement of the above preparation method, in the purification of step 2), the organic solvent is selected from methanol, ethanol, acetone, acetonitrile or mixture thereof; the volume percentage of the organic solvent is in a range from 1% to 90%, and the ratio of optimal organic solvent is in a range from 1% to 50%. The pH of the purified collection solution needs to be adjusted to 1.0 to 6.0, and the optimal pH is in a range from 3.0 to 4.0. During purification and separation, the temperature is controlled at 10° C. to 30° C. The optimal temperature is in a range from 15° C. to 25° C.

The organic solvent used in preparation step (c) is selected from one or a combination of toluene, N,N-dimethylformamide (DMF), N,N-dimethylacetamide (DME), dimethyl sulfoxide (DMSO), tetrahydrofuran (THF), and dichloromethane. An optional solvent is N,N-dimethylformamide (DMF). In step (c), the purification is performed using ion exchange resin, macroporous adsorption resin, and silica gel for purification in sequence.

As a further improvement of the above preparation method, the purification using silica gel specifically comprises the following steps:
(1) diluting the concentrate obtained and preparing a sample solution, and adsorbing it through a silica gel column; and
(2) eluting using an acidic organic solvent aqueous solution.

The silica gel is silica gel with hydrophilic interaction chromatography mode (HILIC). The proportion of organic solvent in the sample solution of step (1) does not exceed 5%, and the silica gel is selected from at least one of UniSil® HILIC silica gel (nano micro), Chromatorex ARG Silica Fuji, Click XIon silica gel (Huapu Xinchuang), and Inertsil HILIC silica gel (Shimadzu, Japan). As a further improvement of the above purification method, the organic solvent is selected from one or a combination of methanol, ethanol, acetone, and acetonitrile. The volume percentage of organic solvents and water used is in a range from 20% to 100%, with an optional range in a range from 50% to 100%.

As a further improvement of the above purification method, after purification through a chromatography column, the HPLC purity of the obtained echinocandin drug impurity is 90% or more. The collection solution components are concentrated below 20° C. and freeze-dried to obtain the finished product of echinocandin drug impurity.

The high-purity echinocandin drug impurity with the Formula I structure can be used in the quality control of echinocandin drugs and used as reference substance for establishing analytical methods. High-purity echinocandin drug impurity refers to its HPLC purity is 90% or more.

Compared with the prior art, the beneficial effects of the present application are:

In the preparation method of the present application, the FR901379 fermentation broth containing impurities of Formula II is extracted and enriched; the compound of Formula III is obtained by conversion and separation using acyltransferase; and a high-purity echinocandin drug impurity (Formula I) is obtained by further preparation.

The present application also provides a further purification method. During the purification process, purification is performed using ion exchange resin, macroporous adsorption resin and silica gel column in sequence, so that the HPLC purity of the echinocandin drug impurity in the reaction solution is further improved, and the HPLC purity reaches a maximum of 90% or more. The above provides new ideas for industrial production. It is a well-known requirement in the art that the purity of drug impurity reference substance must reach 90% or more. Through the present application, echinocandin drug impurity with a purity of 90% or more has been prepared for the first time and can therefore be used as a reference substance for drug quality control.

### BRIEF DESCRIPTION OF THE DRAWINGS

FIG. 1 is a nuclear magnetic resonance hydrogen spectrum (1H NMR) of the echinocandin drug impurity in Example 2.

FIG. 2 is a nuclear magnetic resonance carbon spectrum (13C NMR) of the echinocandin drug impurity in Example 2.

FIG. 3 is a high-resolution mass spectrum (HRMS) of echinocandin drug impurity in Example 2.

FIG. 4 is the HPLC spectrum of echinocandin drug impurity in Example 2.

### DETAILED DESCRIPTION

In order to better illustrate the purpose, technical solution, and advantages of this application, further explanation will be provided below in conjunction with specific embodiments. Those skilled in the art should understand that the specific embodiments described here are only intended to explain the present application and are not intended to limit the present application.

Unless otherwise specified, the test methods used in the examples are conventional methods; unless otherwise specified, the materials and reagents used can be obtained from commercial sources. The fermentation strain Coleophoma sp used in each example was from Advanced Industrial Science and Technology.

### Example 1

### Step 1

Referring to the patent CN102618604B (Shanghai Techwell), Coleophoma sp was used as the fermentation strain. The main components of the culture medium comprise cottonseed powder, mannitol, sodium glutamate, and proline. It was cultured at a temperature ranging from 23° C. to 28° C. for 6 days to 8 days to obtain 5 L of fermentation broth containing FR901379 impurity component, converted into about 20 g of pure FR901379, 5 L methanol was added for extraction, stirred for 2 hours and mixed evenly, then filtered. The filtrate was diluted with water to an organic solvent ratio of 5%, then HP20 resin was used for adsorption, and 80% ethanol was used for elution. After enrichment, a collection solution containing FR901379 impurity (Formula II structure) was obtained. The Formula II structure was about 0.7 g (convert into purification).

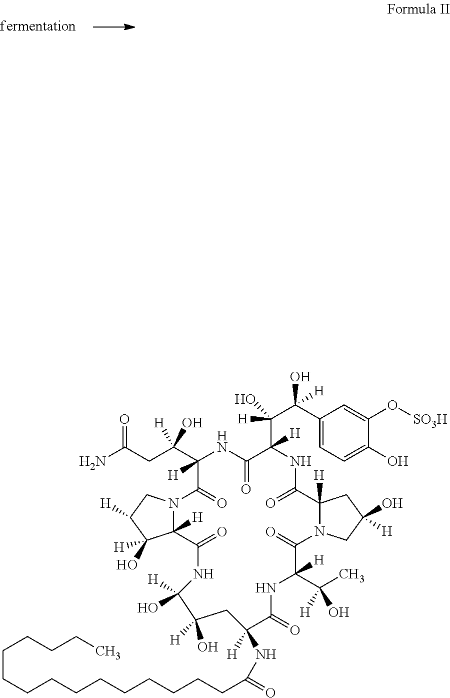

### Step 2

2 L of pH 6.0 phosphate buffer solution were slowly add to the collection solution in step 1, then 15 g of immobilized acyltransferase were added, and the reaction was carried out at 40° C. for 2 hours. SP207SS (1 L) macroporous adsorption resin was added for adsorption, and then eluted sequentially with 1% ethanol and 5% ethanol under controlled temperature ranging from 10° C. to 15° C. The effluent containing FR179642 impurity (Formula III structure) was collected and the pH was adjusted to 3.0. 0.4 g of the compound of Formula III with a purity of 90% was obtained after nanofiltration and freeze-drying.

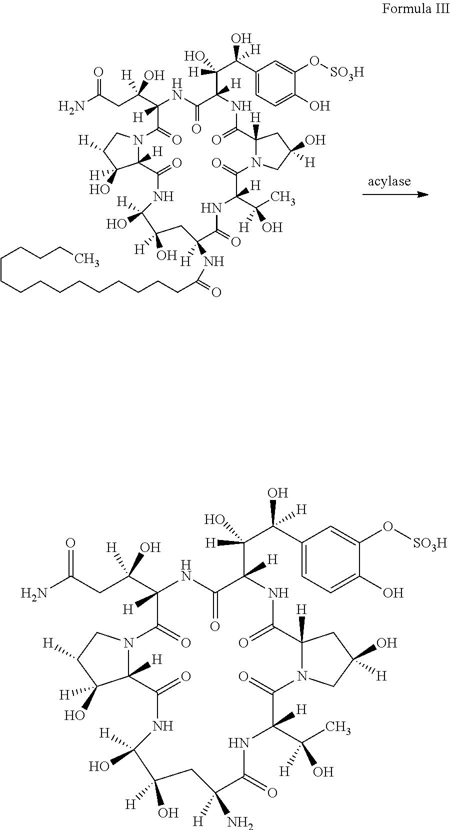

### Step 3

19 g of DMF was added to a 100 mL reaction bottle, cooled to −20° C., and stirred for 5 minutes. 1.0 g of the compound of Formula III structure was added, stirred until clear, 0.22 g of DIPEA and 1.00 g of micafungin sodium side chain were added in sequence at −20° C., and kept warm and reacted for 1.5 hours. The temperature was controlled at 5° C., 4 g methanol was added, stirred for 20 minutes, then 23 g acetone was added and stirred for 1 hour. Finally, 80 g ethyl acetate was dropped at about 1 hour, and a white solid precipitated. The reaction solution was centrifuged to obtain 1.5 g of crude micafungin sodium drug impurity product.

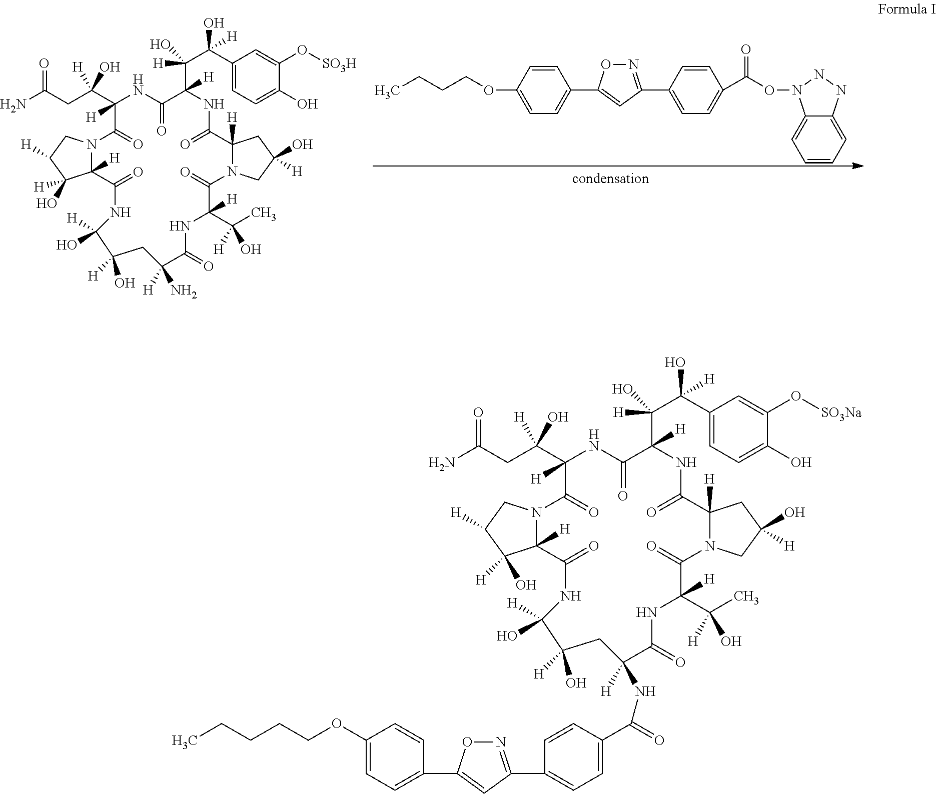

1.5 g of crude micafungin sodium drug impurity was dissolved in 50 ml of water, UBK550 resin (50 ml) was used for ion exchange, and 0.2M sodium bicarbonate was used to adjust the pH of the collection solution containing impurity components to about 5.5. After enrichment using HP20 resin, 80% ethanol aqueous solution was used for elution to obtain a combined solution with a purity of 80% or more. Finally, UniSil® HILIC silica gel was used for purification, and 50% ethanol formic acid aqueous solution (pH 4.0) was used for elution. Components with purity of 90% or more were collected, concentrated and freeze-dried to obtain 0.5 g of micafungin drug impurity (Formula I) as white powder with a purity of 92%.

### Example 2

### Step 1

Referring to the patent CN102618604B (Shanghai Techwell), Coleophoma sp was used as the fermentation strain. The main components of the culture medium comprise cottonseed powder, mannitol, sodium glutamate, and proline. It was cultured at a temperature ranging from 23° C. to 28° C. for 6 days to 8 days to obtain 5 L of fermentation broth containing FR901379 impurity component, converted into about 20 g of pure FR901379, 5 L of acetone was added for extraction, stirred for 30 minutes and mixed evenly, then filtered. The filtrate was diluted with water to an organic solvent ratio of 40%, then HP20SS resin was used for adsorption, and 80% ethanol was used for elution. After enrichment, a collection solution containing FR901379 impurity (Formula II structure) was obtained. The Formula II structure was about 1.3 g (convert into purification).

### Step 2

The collection solution in step 1 containing the FR901379 impurity (Formula II structure) was added 2 L of buffer (0.2M potassium dihydrogen phosphate-disodium hydrogen phosphate buffer; pH 6.0) and 15 g of acyl transfer immobilized enzyme were slowly added, and the reaction was carried out at 40° C. for 2 hours. SP207SS (1 L) macroporous adsorption resin was used for adsorption, washed with 1% ethanol and then eluted with 5% ethanol under controlled temperature ranging from 10° C. to 15° C. The effluent containing FR179642 impurity (Formula III structure) was collected and the pH was adjusted to 3.5. 1.0 g of the compound of Formula III with a purity of 93% was obtained after nanofiltration and freeze-drying.

### Step 3

19 g of DMF was added to a 100 mL reaction bottle, cooled to 0° C., and stirred for 5 minutes. 1.0 g of the compound of Formula III structure was added, stirred until clear, 0.22 g DIPEA and 1.00 g of micafungin sodium side chain were added in sequence at −5° C. The temperature was controlled at 5° C., kept warm and reacted for 1.5 hours, 4 g methanol was added at a controlled temperature of 10° C., stirred for 20 minutes, then 23 g of acetone was added and stirred for 1 hour. Finally, 80 g of ethyl acetate was dropped at about 1 hour, and a white solid precipitated. The reaction solution was centrifuged to obtain 1.5 g of crude micafungin sodium drug impurity product.

1.5 g of crude micafungin sodium drug impurity was dissolved in 50 ml of water, UBK530 resin (50 ml) was used for ion exchange, and 0.2M sodium bicarbonate was used to adjust the pH of the collection solution containing impurity components to about 5.5. After enrichment using HP20SS resin, 60% methanol aqueous solution was used for elution to obtain a combined solution with a purity of 80% or more. Finally, Click XiON silica gel was used for purification, and 60% methanol hydrochloric acid aqueous solution (pH 4.0) was used for elution. Components with purity of 90% or more were collected, concentrated and freeze-dried to obtain 1.0 g of micafungin drug impurity (Formula I) as white powder with a purity of 96.14%.

### Example 3

### Step 1

Referring to the patent CN102618604B (Shanghai Techwell), Coleophoma sp was used as the fermentation strain. The main components of the culture medium comprise cottonseed powder, mannitol, sodium glutamate, and proline. It was cultured at a temperature ranging from 23° C. to 28° C. for 6 days to 8 days to obtain 5 L of fermentation broth containing FR901379 impurity component, converted into about 20 g of pure FR901379, 3 L of propanol was added for extraction, stirred for 30 minutes and mixed evenly, then filtered. The filtrate was diluted with water to an organic solvent ratio of 30%, then XAD-1 resin was used for adsorption, and 65% methanol was used for elution. After enrichment, a collection solution containing FR901379 impurity (Formula II structure) was obtained. The Formula II structure was about 1.2 g (convert into purification).

### Step 2

The aqueous solution containing the FR901379 impurity (Formula II structure) collected in step 1 above was diluted to 2 L, 1 L of pH 6.0 phosphate buffer and 10 g of immobilized acyltransferase were slowly added, and the reaction was carried out at 40° C. for 2 hours. SP207SS (1 L) macroporous adsorption resin was used for adsorption, the temperature was control at 30° C., 2% ethanol was used for washing, and then 30% acetonitrile was used for elution. The effluent containing FR179642 impurity (Formula III structure) was collected and the pH was adjusted to 4.5. 0.3 g of the compound of Formula III with a purity of 93% was obtained after nanofiltration and freeze-drying.

### Step 3

10 ml of DMSO was added to a 100 mL reaction bottle, cooled to −20° C., and stirred for 30 minutes. 0.3 g of the compound of Formula III structure was added, stirred until clear, 0.1 g of DIPEA and 0.2 g of micafungin sodium side chain were added in sequence at −10° C., the temperature was controlled at 20° C., and kept warm and reacted for 1.5 hours. 4 g ethanol was added at a controlled temperature of 0° C., stirred for 20 minutes. Finally, 30 g of ethyl acetate was dropped at about 1 hour, and a white solid precipitated. The reaction solution was centrifuged to obtain 0.3 g of crude micafungin sodium drug impurity product.

0.3 g of crude micafungin sodium drug impurity was dissolved in 10 ml of water, UBK550 resin (50 ml) was used for ion exchange, and 0.2M sodium bicarbonate was used to adjust the pH of the collection solution containing impurity components to about 5.5. After enrichment using HP20 resin, 99% methanol aqueous solution was used for elution to obtain a combined solution with a purity of 80% or more. Finally, Chromatorex ARG Silica Fuji silica gel was used for purification, and 70% acetonitrile trifluoroacetic acid aqueous solution (pH 5.0) was used for elution. Components with purity of 90% or more were collected, concentrated and freeze-dried to obtain 2.0 g of micafungin drug impurity (Formula I) as white powder with a purity of 95%.

### Example 4

### Step 1

Referring to the patent CN102618604B (Shanghai Techwell), Coleophoma sp was used as the fermentation strain. The main components of the culture medium comprise cottonseed powder, mannitol, sodium glutamate, and proline. It was cultured at a temperature ranging from 23° C. to 28° C. for 6 days to 8 days to obtain 20 L of fermentation broth containing FR901379 impurity component, converted into about 40 g of pure FR901379, 10 L of butanone/ethanol mixed solution (1:1) was added for extraction, stirred for 2 hours and mixed evenly, then filtered. The filtrate was diluted with water to an organic solvent ratio of 30%, then HP30 resin was used for adsorption, and 90% acetonitrile was used for elution. After enrichment, a collection solution containing FR901379 impurity (Formula II structure) was obtained. The Formula II structure was about 2.0 g (convert into purification).

### Step 2

The aqueous solution containing the FR901379 impurity (Formula II structure) collected in step 1 above was diluted to 2 L, 1 L of pH 6.0 phosphate buffer and 20 g of immobilized acyltransferase were slowly added, and the reaction was carried out at 40° C. for 3 hours. Unips10 (1.5 L) resin was used for adsorption, the temperature was control at 20° C., 20% acetonitrile was used for washing, and then 65% acetonitrile was used for elution. FR179642 impurity (Formula III structure) was collected and the pH was adjusted to 3.0. 1.0 g of the compound of Formula III with a purity of 80% was obtained after nanofiltration and freeze-drying.

### Step 3

20 ml of THF was added to a 100 mL reaction bottle, cooled to −18° C., and stirred for 1 hour. 1.0 g of the compound of Formula III structure was added, stirred until clear, 0.1 g of triethylamine and 0.5 g of micafungin sodium side chain were added in sequence at −10° C., the temperature was controlled at 5° C., kept warm and reacted for 1.5 hours. 2 g of methanol was added, stirred for 20 minutes. Finally, 30 ml of ethyl acetate was dropped at about 1 hour, and a white solid precipitated, which was centrifuged to obtain 0.8 g of crude micafungin sodium drug impurity product.

0.8 g of crude micafungin sodium drug impurity was dissolved in 10 ml of water, UBK530 resin (50 ml) was used for ion exchange, and 0.2M sodium hydroxide was used to adjust the pH of the collection solution containing impurity components to about 5.5. After enrichment using CHP20 resin, 50% methanol aqueous solution was used for elution to obtain a combined solution with a purity of 90% or more. Finally, Inertsil HILIC silica gel was used for purification, and 50% methanol trifluoroacetic acid water (pH 6.0) was used for elution. Components with purity of 90% or more were collected, concentrated and freeze-dried to obtain 0.6 g of micafungin drug impurity (Formula I) as white powder with a purity of 95.8%.

### Example 5

### Step 1

Referring to the patent CN102618604B (Shanghai Techwell), Coleophoma sp was used as the fermentation strain. The main components of the culture medium comprise cottonseed powder, mannitol, sodium glutamate, and proline. It was cultured at a temperature ranging from 23° C. to 28° C. for 6 days to 8 days to obtain 30 L of fermentation broth containing FR901379 impurity component, converted into about 50 g of pure FR901379, 30 L of methanol/ethanol mixed solution (1:1) was added for extraction, stirred for 2 hours and mixed evenly, then filtered. The filtrate was diluted with water to an organic solvent ratio of 50%, then CHP20 resin was used for adsorption, and 90% acetonitrile was used for elution. After enrichment, a collection solution containing FR901379 impurity (Formula II structure) was obtained. The Formula II structure was about 2.0 g (convert into purification).

### Step 2

The aqueous solution containing the FR901379 impurity (Formula II structure) collected in step 1 above was diluted to 2 L, 1 L of pH 6.0 phosphate buffer and 20 g of immobilized acyltransferase were slowly added, and the reaction was carried out at 40° C. for 3 hours. UniPS50 (3 L) resin was used for adsorption, the temperature was control at 22° C., 30% acetonitrile was used for washing, and then 70% acetonitrile was used for elution. The effluent containing FR179642 impurity (Formula III structure) was collected and the pH was adjusted to 6.0. 1.1 g of the compound of Formula III with a purity of 86% was obtained after nanofiltration and freeze-drying.

### Step 3

20 ml of DMF was added to a 100 mL reaction bottle, cooled to −15° C., and stirred for 1 hour. 1.1 g of the compound of Formula III structure was added, stirred until clear, 0.1 g triethylamine and 0.6 g of micafungin sodium side chain were added in sequence at −10° C., kept warm at 0° C. and reacted for 1.5 hours. 50 ml of ethyl acetate was dropped in and a white solid precipitated, which was filtrated and dried to obtain 0.9 g of crude micafungin sodium drug impurity.

0.9 g of crude micafungin sodium drug impurity was dissolved in 10 ml of water, UBK530 resin (50 ml) was used for ion exchange, and 0.2M sodium hydroxide was used to adjust the pH of the collection solution containing impurity components to about 5.5. After enrichment using HP20SS resin, 80% methanol aqueous solution was used for elution to obtain a combined solution with a purity of 90% or more. Finally, Chromatorex ARG Silica Fuji silica gel was used for purification, and 50% methanol trifluoroacetic acid water (pH 6.0) was used for elution. Components with purity of 90% or more were collected, concentrated and freeze-dried to obtain 0.6 g of micafungin drug impurity (Formula I) as white powder with a purity of 98.1%.

Sample prepared in Example 2 was taken for analysis:

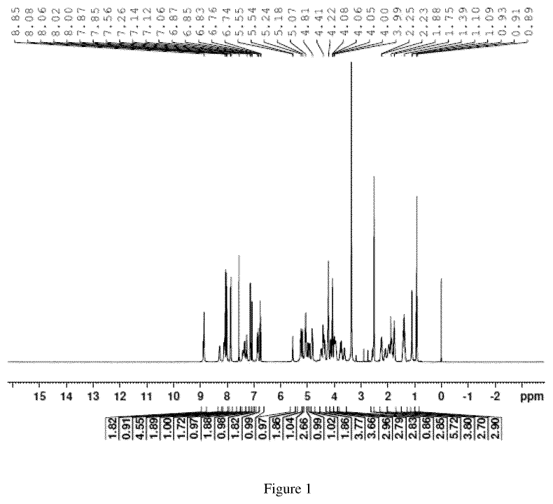

FIG. 1 is a nuclear magnetic resonance hydrogen spectrum (1H NMR) of the echinocandin drug impurity in Example 2. The results were as follows:

1H NMR (DMSO-_{d6}, 400 MHz) δ: 0.91 (t, J=8.0 Hz, 3H), 1.09 (d, J=8.0 Hz, 3H), 1.35-1.45 (m, 4H), 1.75-2.08 (m, 6H), 2.04-2.10 (m, 1H), 2.20-2.25 (m, 2H), 2.53-2.60 (m, 1H), 3.60-3.75 (m, 3H), 3.88-3.97 (m, 3H), 4.08 (t, J=8 Hz, 2H), 4.14 (d, J=12 Hz, 1H), 4.19-4.23 (m, 4H), 4.30-4.37 (m, 3H), 4.49-4.51 (m, 1H), 4.78-4.81 (m, 2H), 4.92-4.99 (m, 2H), 5.04-5.08 (m, 3H), 5.18 (J=8.0 Hz, 1H), 5.20-5.24 (m, 2H), 5.55 (d, J=8.0 Hz, 1H), 6.75 (d, J=12.0 Hz, 2H), 6.83-6.87 (m, 2H), 7.06 (d, J=8.0 Hz, 1H), 7.13 (d, J=8.0 Hz, 2H), 7.26 (b, 1H), 7.26 (b, 1H), 7.34 (d, J=8.0 Hz, 1H), 7.39 (b, 1H), 7.56 (s, 1H), 7.86 (d, J=8.0 Hz, 2H), 8.01 (d, J=8.0 Hz, 2H), 8.06-8.12 (m, 3H), 8.27 (d, J=8.0 Hz, 1H), 8.85 (s, 1H), 8.90 (d, J=8.0 Hz, 1H).

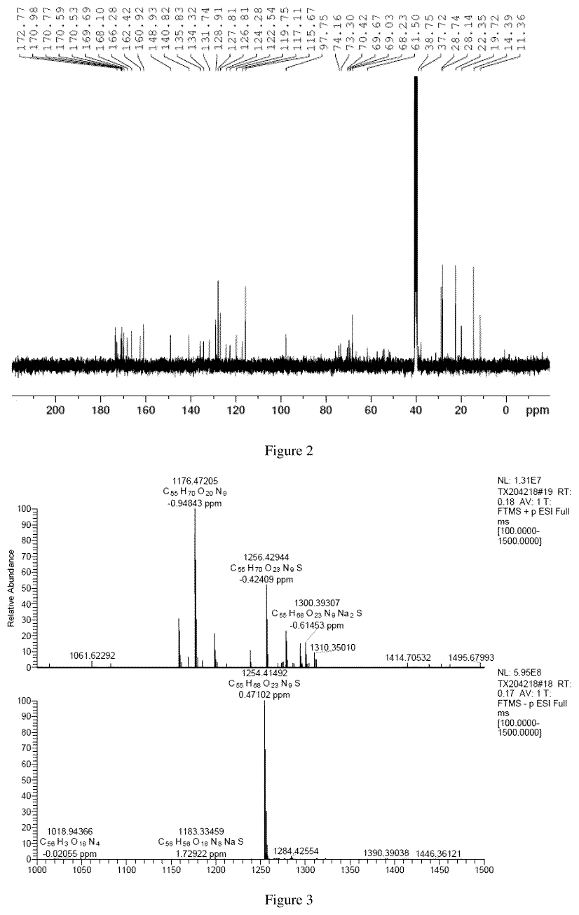

FIG. 2 is a nuclear magnetic resonance carbon spectrum (13C NMR) of the echinocandin drug impurity in Example 2. The results were as follows:

13C NMR (DMSO-_{d6}, 400 MHz) δ: 14.39, 19.76, 22.35, 28.13, 28.74, 33.77, 34.01, 37.84, 38.75, 45.73, 51.52, 54.03, 54.51, 56.30, 57.34, 61.35, 66.79, 68.23, 68.50, 69.66 69.66, 69.86, 72.27, 73.01, 73.31, 75.51, 97.75, 115.67, 117.12, 119.75, 122.52, 124.24, 126.82, 127.80, 128.90, 131.79, 134.30, 135.64, 140.84, 148.95, 160.93, 162.40, 166.09, 168.34, 169.91, 170.47, 170.60, 170.94, 170.94, 172.09, 173.35.

FIG. 3 is a high-resolution mass spectrum (HRMS) of echinocandin drug impurity in Example 2, HRMS (ESI): calcd. For C₅₅H₆₈N₉O₂₃Na₂S (M+Na+): 1300.3930, Found: 1300.3939.

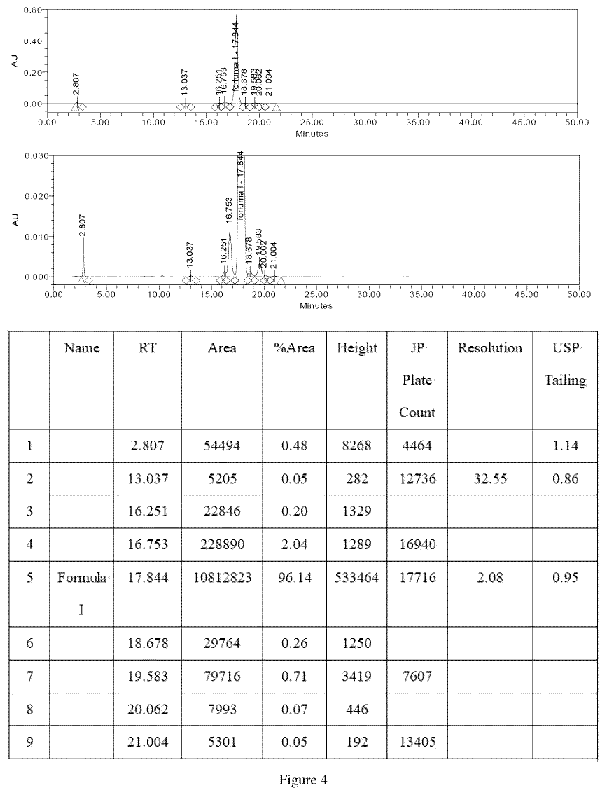

FIG. 4 is the HPLC spectrum of echinocandin drug impurity in Example 2.

Based on the above test results, it can be seen that high-purity echinocandin drug impurity represented by Formula I were prepared in Example 2.

The above detection methods were used to detect the products of the remaining examples, and the test results showed that high-purity echinocandin drug impurity with a purity of greater than 90% were prepared in each example.

### Comparative Example 1

### Step 1

Referring to the patent CN102618604B (Shanghai Techwell), Coleophoma sp was used as the fermentation strain. The main components of the culture medium comprise cottonseed powder, mannitol, sodium glutamate, and proline. It was cultured at a temperature ranging from 23° C. to 28° C. for 6 days to 8 days to obtain 30 L of fermentation broth containing FR901379 impurity component, converted into about 50 g of pure FR901379. It was directly filtered and concentrated at 10° C. to obtain a collection solution containing the FR901379 impurity (Formula II structure). The Formula II structure was about 0.5 g (convert into purification).

### Step 2

The aqueous solution containing the FR901379 impurity (Formula II structure) collected in step 1 above was diluted to 2 L, 1 L of pH 7.0 phosphate buffer and 20 g of immobilized acyltransferase were slowly added, and reacted at 40° C. for 3 hours. UniPS50 (3 L) resin was used for adsorption. At 31° C., 30% acetonitrile was used for washing, and then 70% acetonitrile was used for elution. Effluent containing FR179642 impurities (Formula III structure) was collected and pH was adjusted to 7.0. 0.3 g of the compound of Formula III with a purity of 50% was obtained after nanofiltration and freeze-drying.

### Step 3

10 ml of DMF was added to the 100 mL reaction bottle, cooled to −15° C., and stirred for 1 hour. 0.1 g of the compound of Formula III structure was added, stirred until clear, 0.1 g of triethylamine and 0.6 g of micafungin sodium side chain were added in sequence at −10° C., the temperature was control at 0° C. to 5° C., and kept warm and reacted for 1.5 hours. 50 ml of ethyl acetate was dropped in and a white solid precipitated, which was filtered and dried to obtain 0.4 g of crude micafungin sodium drug impurity.

0.4 g of crude micafungin sodium drug impurity was dissolved in 10 ml of water, UBK530 resin (50 ml) was used for ion exchange, and 0.2M sodium hydroxide was used to adjust the pH of the collection solution containing impurity component to 7.0. After enrichment using HP20SS resin, 80% methanol aqueous solution was used for elution to obtain a combined solution. Finally, Click XiON silica gel was used for purification, and 60% methanol hydrochloric acid aqueous solution (pH 4.0) was used for elution. The component that mainly contains micafungin sodium pharmaceutical impurity was collected, concentrated at 25° C. and freeze-dried to obtain 0.11 g of pure micafungine drug impurity (Formula I) with a purity of 52%.

### Comparative Example 2

### Step 1

Referring to the patent CN102618604B (Shanghai Techwell), Coleophoma sp was used as the fermentation strain. The main components of the culture medium comprise cottonseed powder, mannitol, sodium glutamate, and proline. It was cultured at a temperature ranging from 23° C. to 28° C. for 6 days to 8 days to obtain 5 L of fermentation broth containing FR901379 impurity component, converted into about 20 g of pure FR901379. 30 L of methanol/ethanol mixed solution (1:1) was added for extraction, stirred for 2 hours and mixed evenly, then filtered. The filtrate was diluted with water to an organic solvent ratio of 50%, then CHP20 resin was used for adsorption, and 90% acetonitrile was used for elution. After enrichment, a collection solution containing FR901379 impurity (Formula II structure) was obtained. The Formula II structure was about 2.0 g (convert into purification).

### Step 2

The aqueous solution containing the FR901379 impurity (Formula II structure) collected in the above step 1 was diluted to 2 L, and sodium bicarbonate was slowly added for hydrolysis. No compound of Formula III structure was detected by HPLC.

### Comparative Example 3

### Step 1

Referring to the patent CN102618604B (Shanghai Techwell), Coleophoma sp was used as the fermentation strain. The main components of the culture medium comprise cottonseed powder, mannitol, sodium glutamate, and proline. It was cultured at a temperature ranging from 23° C. to 28° C. for 6 days to 8 days to obtain 5 L of fermentation broth containing FR901379 impurity component, converted into about 20 g of pure FR901379. 30 L of methanol/ethanol mixed solution (1:1) was added for extraction, stirred for 2 hours and mixed evenly, then filtered. The filtrate was diluted with water to an organic solvent ratio of 50%, then CHP20 resin was used for adsorption, and 90% acetonitrile was used for elution. After enrichment, a collection solution containing FR901379 impurity (Formula II structure) was obtained. The Formula II structure was about 2.0 g (convert into purification).

### Step 2

The aqueous solution containing the FR901379 impurity (Formula V structure) collected in step 1 above was diluted to 2 L, 1 L of pH 7.0 phosphate buffer and 20 g of immobilized acyltransferase were slowly added, and the reaction was carried out at 40° C. for 3 hours. UniPS50 (3 L) resin was used for adsorption, 30% acetonitrile was used for washing at 20° C., and then 70% acetonitrile was used for elution. FR179642 impurity (Formula III structure) was collected and the pH was adjusted to 6.0. 1.1 g of the compound of Formula III with a purity of 85% was obtained after nanofiltration and freeze-drying.

### Step 3

20 ml of DMF was added to the 100 mL reaction bottle, cooled to −15° C., and stirred for 1 hour. 1.1 g of the compound of Formula III structure was added, stirred until clear, 0.2 g of triethylamine and 0.6 g of micafungin sodium side chain were added in sequence at −10° C., kept warm at 0° C. to 5° C. and reacted for 1.5 hours. 50 ml of ethyl acetate was dropped in and a white solid precipitated, which was centrifuged to obtain 0.4 g of crude micafungin sodium drug impurity.

0.4 g of crude micafungin sodium drug impurity was dissolved in 10 ml of water, Click XiON silica gel was used for purification, and 60% methanol aqueous solution was used for elution. The component that mainly contain micafungin sodium drug impurity was collected, concentrated at 30° C., and freeze-dried to obtain 0.1 g of pure micafungin drug impurity (Formula I) as light yellow powder with a purity of 47%.

Obviously, the above examples are merely examples made for clear description, rather limiting the implementations. For those of ordinary skill in the art, other different forms of variations or modifications can also be made on the basis of the above-mentioned description. All embodiments are not necessary to be and cannot be exhaustively listed herein. In addition, obvious variations or modifications derived therefrom all fall within the scope of protection of the present invention.

1. An echinocandin drug impurity, wherein a structure of the echinocandin drug impurity is shown in Formula I, and the echinocandin drug impurity has a purity of greater than 90%;

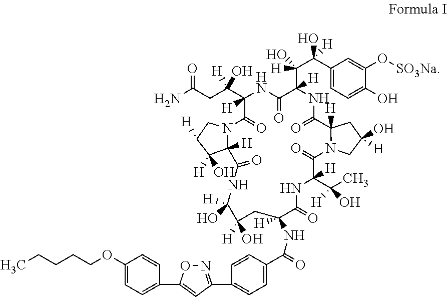

2. A method for preparing the echinocandin drug impurity of claim 1, wherein the method comprises the following steps:
step 1) adding an organic solvent to a microbial fermentation broth containing a compound of Formula II, mixing well and then filtering to obtain a filtrate, and enriching the filtrate by using a first adsorption resin to obtain the compound of Formula II; 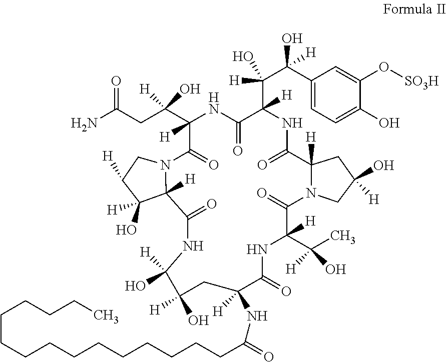
step 2) reacting the enriched compound of Formula II with an acyltransferase to obtain a compound of Formula III; 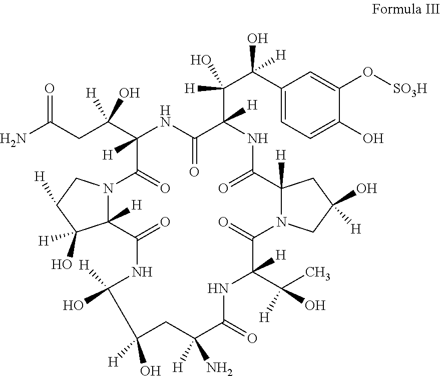
step 3) reacting the compound of Formula III obtained with the side chain of micafungin sodium to obtain the echinocandin drug impurity.

3. The method for preparing the echinocandin drug impurity of claim 2, wherein the microbial fermentation broth in step 1) is obtained by fermentation of Coleophoma sp.;
optionally, the organic solvent is selected from at least one of methanol, ethanol, propanol, acetone and butanone;
optionally, a volume ratio of the organic solvent to the microbial fermentation broth is in a range from 1% to 40%.

4. The method for preparing the echinocandin drug impurity of claim 2, wherein the first adsorption resin is a non-polar aromatic adsorption resin formed by polymerization of styrene and divinylbenzene;
optionally, the first adsorption resin is at least one of AD-1, HP20, HP20SS, HP30, SP-825, SP-850, SP207 and CHP20.

5. The method for preparing the echinocandin drug impurity of claim 2, wherein the reacting the enriched compound of Formula II with an acyltransferase in step 2) further comprises a purification step, and the purification step comprises adsorption using a second adsorption resin, and elution using an organic solvent aqueous solution at a temperature ranging from 10° C. to 30° C.

6. The method for preparing the echinocandin drug impurity of claim 5, wherein the second adsorption resin is a macroporous adsorption resin, and at least one of the resins SP207, SP207SS, UniPS10, UniPS20, and UniPS50 using styrene and divinylbenzene as matrix is used;
optionally, the organic solvent in the organic solvent aqueous solution is selected from at least one of methanol, ethanol, acetone and acetonitrile;
optionally, the volume percentage of organic solvent in the organic solvent aqueous solution is in a range from 1% to 90%;
optionally, the elution is performed at a temperature in a range from 15° C. to 25° C.;
optionally, the acyltransferase is a carrier-loaded immobilized acyltransferase.

7. The method for preparing the echinocandin drug impurity of claim 2, wherein the compound of Formula III is reacted with the side chain of micafungin sodium at a temperature ranging from −20° C. to 10° C.;
optionally, the compound of Formula III is reacted with the side chain of micafungin sodium in an organic solvent, and the organic solvent is selected from at least one of toluene, N,N-dimethylformamide, N,N-dimethylethyl amide, dimethyl sulfoxide, tetrahydrofuran and methylene chloride.

8. The method for preparing the echinocandin drug impurity of claim 2, wherein, performing a purification after the reaction of compound of Formula III and micafungin sodium complete, and the purification is performed by using ion exchange resin, macroporous adsorption resin, and silica gel in sequence;
optionally, the ion exchange resin is a strongly acidic cation exchange resin, and the ion exchange resin is selected from at least one of UBK530, UBK550 and UBK555;
the macroporous adsorption resin is selected from at least one of HP20, HP20SS, SP70, SP700, SP825L, SP850, CHP20 and CHP55;
the silica gel is selected from at least one of pure silica gel, amino-bonded silica gel, diol-bonded silica gel and amide-bonded silica gel.

9. The method for preparing the echinocandin drug impurity of claim 8, wherein the purification using silica gel comprises adsorption using a silica gel column, and then elution using an acidic organic solvent aqueous solution with a pH value ranging from 2.0 to 7.0;
optionally, the volume percentage of organic solvent and water in the acidic organic solvent aqueous solution is in a range from 20% to 100%;
optionally, the organic solvent in the acidic organic solvent aqueous solution is selected from one of methanol, ethanol, acetone and acetonitrile;
optionally, the acidic organic solvent aqueous solution contains at least one of formic acid, acetic acid, hydrochloric acid, phosphoric acid and trifluoroacetic acid;
optionally, the pH value of the acidic organic solvent aqueous solution is in a range from 3.0 to 5.0.

10. A method for quality control of echinocandin drugs, wherein the echinocandin drug impurity of claim 1 is used as a reference substance.
